(19) United States
(12) Patent Application Publication
(10) Pub. No.: US 2023/0390553 A1
(43) Pub. Date: Dec. 7, 2023

(54) MULTIFUNCTIONAL BEAUTY DEVICE AND HANDPIECE OF BEAUTY INSTRUMENT

(71) Applicant: SHANGHAI CICI BEAUTY AND SLIM INDUSTRIAL CO., LTD., Shanghai (CN)

(72) Inventor: Yongxing YAN, Shanghai (CN)

(73) Assignee: SHANGHAI CICI BEAUTY AND SLIM INDUSTRIAL CO., LTD., Shanghai (CN)

(21) Appl. No.: 18/032,356

(22) PCT Filed: Oct. 18, 2020
(86) PCT No.: PCT/CN2020/121736

§ 371 Date: Apr. 18, 2023

(51) Int. Cl. A61N 1/32; A61B 18/12; A61H 9/00

(52) U.S. Cl. CPC A61N 1/32; A61B 18/12; A61H 9/0057

(57) ABSTRACT

A multifunctional beauty device includes: a base, where one surface of the base is provided with an annular separator plate, and a negative pressure cavity is formed between the annular separator plate and the base; a negative pressure air hole communicating with the negative pressure cavity and configured to connect a negative pressure generator; a plurality of bipolar contact electrodes arranged on one side of the annular separator plate and connected to the base; a plurality of microcurrent electrodes arranged on one side of the annular separator plate and connected to the base; a massage post arranged on one side of the annular separator plate and connected to the base; and a heating module arranged on the other surface of the base opposite to the annular separator plate and configured to preheat the bipolar contact electrodes, the microcurrent electrodes and the massage post.

### CROSS REFERENCE TO THE RELATED APPLICATIONS

This application is the national phase entry of International Application No. PCT/CN2020/121736, filed on Oct. 18, 2020, the entire contents of which are incorporated herein by reference.

### TECHNICAL FIELD

The present disclosure relates to the technical field of beauty devices, and in particular to a multifunctional beauty device and a handpiece of a beauty instrument.

### BACKGROUND

Beauty physiotherapy is a method for preventing and treating diseases by applying artificial or natural physical factors to human bodies. It is an important content of rehabilitation therapy. Cosmetology is an ancient and young discipline. It treats diseases and maintains the health with sunlight, stone needles, water, massages, etc. According to early literatures, the diseases are prevented and treated not only by sunlight, hot bathes, cold baths, exercises and massages, but also by physical beauty therapies such as acupuncture, cupping, thermotherapy, breathing training, massotherapy, and hydrotherapy.

Prior beauty instruments are short for physical beauty instruments. They are intended to apply physical factors to human bodies for a desired effect, and are applied to homes, offices, etc. Common physical factors include electricity, sound, light, magnetism, water, pressure and so forth. In contrast with the drug therapy, the beauty physiotherapy is safer and more environment-friendly. However, most of prior beauty devices have a single function and can only solve problems locally. For example, they can only be used to conduct microcurrent stimulation, heating, cupping or the like, and cannot realize various beauty operations at the same time. Therefore, it is desired to provide a multifunctional beauty device capable of synthesizing various functional modules.

### SUMMARY

An objective of the present disclosure is to provide a multifunctional beauty device and a handpiece of a beauty instrument. The device has more functions, and can conduct various beauty operations. Moreover, various functional modules can be synthesized, such that the device achieves a stronger universality and a better use effect.

The present disclosure provides the following technical solutions.

The present disclosure provides a multifunctional beauty device, including:

a base, where one surface of the base is provided with an annular separator plate, and a negative pressure cavity is formed between the annular separator plate and the base;
a negative pressure air hole penetrating through the base and communicating with the negative pressure cavity, where the negative pressure air hole is configured to connect a negative pressure generator to form a negative pressure in the negative pressure cavity;
a plurality of bipolar contact electrodes arranged on one side of the annular separator plate and connected to the base, where the bipolar contact electrodes are configured to connect a bipolar radio-frequency (RF) generator;
a plurality of microcurrent electrodes arranged on one side of the annular separator plate and connected to the base, where the microcurrent electrodes are configured to connect a low-intermediate frequency amplitude-modulation synthesizer;
a massage post arranged on one side of the annular separator plate, connected to the base, and configured to conduct a massage when the negative pressure is sucked and released; and
a heating module arranged on the other surface of the base opposite to the annular separator plate and configured to preheat the bipolar contact electrodes, the microcurrent electrodes and the massage post.

The annular separator plate is arranged on the skin contacting base. The negative pressure cavity can be formed between the base and the annular separator plate. The negative pressure air hole connected to the negative pressure generator is provided to form the negative pressure in the negative pressure cavity. Therefore, negative pressure cupping is conducted conveniently. A plurality of bipolar contact electrodes and a plurality of microcurrent electrodes are arranged on a side of the annular separator plate. The bipolar contact electrodes are connected to the bipolar RF generator. The microcurrent electrodes are connected to the low-intermediate frequency amplitude-modulation synthesizer. The bipolar contact electrodes can conduct deep heating on the user for weight reduction. The microcurrent electrodes can conduct microcurrent stimulation on the user for a beauty effect. Meanwhile, with the massage post, when the negative pressure absorptive cupping is conducted, acupoints of the user are squeezed and massaged. With the heating module, before the device is used, the bipolar contact electrodes, the microcurrent electrodes and the massage post can be preheated by the heating module. This improves the body comfort, and prevents the cold metal post from directly contacting the skin of the user to cause the discomfort in a cold period such as winter. According to the above solutions, various beauty functions can be synthesized, and the device achieves more functions, a stronger universality and a better use effect.

Further, the bipolar contact electrodes are arranged on the inner side of the annular separator plate and distributed annularly; and
the bipolar contact electrodes include a plurality of first contact electrodes and a plurality of second contact electrodes, the number of the plurality of first contact electrodes is identical to the number of the plurality of second contact electrodes, and the plurality of first contact electrodes and the plurality of second contact electrodes are symmetric with respect to the center of the negative pressure cavity.

By arranging the bipolar contact electrodes annularly, the bipolar contact electrodes can be used conveniently. Since there is no need to provide too many bipolar contact electrodes, the bipolar contact electrodes can be arranged on the inner side of the annular separator plate.

The bipolar contact electrodes include the plurality of first contact electrodes and the plurality of second contact electrodes, the number of the plurality of first contact electrodes is identical to the number of the plurality of second contact electrodes, and the plurality of first contact electrodes and the plurality of second contact electrodes may be symmetric with respect to the center of the negative pressure cavity. Therefore, the bipolar contact electrodes are heated uniformly.

There are three first contact electrodes and three second contact electrodes in the embodiment. In other embodiments, the number of the first contact electrodes and the second contact electrodes can be adjusted as required in use.

Further, the microcurrent electrodes are arranged on the outer side of the annular separator plate and distributed annularly and uniformly.

Since there are usually many microcurrent electrodes, the microcurrent electrodes can be arranged on the outer side of the annular separator plate. Meanwhile, the microcurrent electrodes are distributed annularly and uniformly, which ensures uniform stimulation of a microcurrent on the skin.

Further, the massage post is arranged in the center of the negative pressure cavity; and
heights of the massage post, the bipolar contact electrodes, the annular separator plate and the microcurrent electrodes gradually increase.

By providing the massage post in the center of the negative pressure cavity, the acupoints of the user can be squeezed and massaged conveniently in negative pressure absorption, and various structures can be distributed on the base more uniformly. The heights of the massage post, the bipolar contact electrodes, the annular separator plate and the microcurrent electrodes gradually increase, which ensures that various functions of the device are not affected to each other, and can be used independently.

Further, the heating module is arranged on the other surface of the base opposite to the annular separator plate, and the base is made of a metal material, such that heat generated by the heating module is transferred to the bipolar contact electrodes, the microcurrent electrodes and the massage post.

Since the massage post is made of metal, when the base is also made of the metal material, the heat generated by the heating module can be transferred to the bipolar contact electrodes, the microcurrent electrodes and the massage post through the base. Therefore, the device can be preheated by the heating module before use, which prevents the cold metal post from directly contacting the skin of the user to cause the discomfort in a cold period such as winter.

Further, a temperature control switch is further arranged between the base and the heating module;
the temperature control switch is fixed on the base through a thermally conductive silicone pad and connected to the heating module; and
the temperature control switch is disconnected when a temperature of the base reaches a preset temperature, such that the heating module stops heating the base.

With the temperature control switch, the heating temperature of the base can be monitored in real time, and the human body is not scalded for an overhigh temperature. The temperature of the temperature control switch is set at 50° C. in the embodiment. In other embodiments, the temperature can be adjusted as required in use.

Further, the bipolar RF generator is connected to a bipolar detection control terminal; and the bipolar detection control terminal is configured to initially control the bipolar RF generator to output second energy to the bipolar contact electrodes, and detect a contact condition between the bipolar contact electrodes and a skin; and
the bipolar detection control terminal controls the bipolar RF generator to output first energy to the bipolar contact electrodes when the bipolar contact electrodes fully contact the skin, where the first energy is greater than the second energy.

The bipolar RF generator is connected to the bipolar detection control terminal, such that the bipolar detection control terminal can monitor the contact condition between the bipolar contact electrodes and the skin in real time. Initially, the bipolar detection control terminal controls the bipolar RF generator to output the second energy to the bipolar contact electrodes. When detecting that the bipolar contact electrodes fully contact the skin, the bipolar detection control terminal receives a signal, and controls the bipolar RF generator to output the first energy to the bipolar contact electrodes. When the bipolar contact electrodes contact the skin poorly, the bipolar detection control terminal does not receive the signal, and controls the bipolar RF generator to continuously output the second energy to the bipolar contact electrodes. The first energy refers to normal output energy, while the second energy is weak energy. The first energy is far greater than the second energy. When the bipolar contact electrodes are used to conduct the deep heating on the user for weight reduction, the contact condition between the bipolar contact electrodes and the skin can be found clearly. This prevents the poor contact between the bipolar contact electrodes and the skin, and can further prevent blind increase of energy output to scald the skin when the bipolar contact electrodes contact the skin poorly.

Additionally, the present disclosure further provides a handpiece of a beauty instrument, including:
a base, where one surface of the base is provided with an annular separator plate, and a negative pressure cavity is formed between the annular separator plate and the base;
a negative pressure air hole penetrating through the base and communicating with the negative pressure cavity, where the negative pressure air hole is configured to connect a negative pressure generator to form a negative pressure in the negative pressure cavity;
a plurality of bipolar contact electrodes arranged on one side of the annular separator plate and connected to the base, where the bipolar contact electrodes are configured to connect a bipolar RF generator;
a plurality of microcurrent electrodes arranged on one side of the annular separator plate and connected to the base, where the microcurrent electrodes are configured to connect a low-intermediate frequency amplitude-modulation synthesizer;
a massage post arranged on one side of the annular separator plate, connected to the base, and configured to conduct a massage when the negative pressure is sucked and released;
a heating module arranged on the other surface of the base opposite to the annular separator plate and configured to preheat the bipolar contact electrodes, the microcurrent electrodes and the massage post;
an upper outer housing connected to the other surface of the base opposite to the annular separator plate and sleeved outside the heating module; and
a handle connected to the upper outer housing.

The annular separator plate is arranged on the skin contacting base. The negative pressure cavity can be formed between the base and the annular separator plate. The negative pressure air hole connected to the negative pressure generator is provided to form the negative pressure in the negative pressure cavity. Therefore, negative pressure cupping is conducted conveniently. A plurality of bipolar contact electrodes and a plurality of microcurrent electrodes are arranged on a side of the annular separator plate. The bipolar contact electrodes are connected to the bipolar RF generator. The microcurrent electrodes are connected to the low-intermediate frequency amplitude-modulation synthesizer. The bipolar contact electrodes can conduct deep heating on the user for weight reduction. The microcurrent electrodes can conduct microcurrent stimulation on the user for a beauty effect. Meanwhile, with the massage post, when the negative pressure absorptive cupping is conducted, acupoints of the user are squeezed and massaged. With the heating module, before the device is used, the bipolar contact electrodes, the microcurrent electrodes and the massage post can be preheated by the heating module. This improves the body comfort, and prevents the cold metal post from directly contacting the skin of the user to cause the discomfort in a cold period such as winter. Additionally, with the upper outer housing and the handle, the device can be used more conveniently. According to the above solutions, various beauty functions can be synthesized, and the device achieves more functions, a stronger universality and a better use effect.

Further, the handle includes a handle seat connected to the upper outer housing, where a grip is arranged on one side of a top of the handle seat, and an arc-shaped gripping member is arranged on the other side of the top of the handle seat; and
through holes are respectively formed in a surface of the upper outer housing and a surface of the handle seat, and the through holes are configured to allow a negative pressure air tube and a line to penetrate through.

By providing the handle seat, and providing the grip and the arc-shaped gripping member on the handle seat, the device can be held conveniently by a single hand or both hands. By forming the through holes in the upper outer housing and the handle seat, the negative pressure air tube connected to the negative pressure air hole and the negative pressure generator, as well as the line connecting the bipolar RF generator and the low-intermediate frequency amplitude-modulation synthesizer, can be provided conveniently.

Further, the bipolar contact electrodes are arranged on the inner side of the annular separator plate and distributed annularly;
the microcurrent electrodes are arranged on the outer side of the annular separator plate and distributed annularly and uniformly;
the massage post is arranged in the center of the negative pressure cavity; and
heights of the massage post, the bipolar contact electrodes, the annular separator plate and the microcurrent electrodes gradually increase.

By arranging the bipolar contact electrodes annularly, the bipolar contact electrodes can be used conveniently. Since there is no need to provide too many bipolar contact electrodes, the bipolar contact electrodes can be arranged on the inner side of the annular separator plate. Since there are usually many microcurrent electrodes, the microcurrent electrodes can be arranged on the outer side of the annular separator plate. Meanwhile, the microcurrent electrodes are distributed annularly and uniformly, which ensures uniform stimulation of a microcurrent on the skin. By providing the massage post in the center of the negative pressure cavity, the acupoints of the user can be squeezed and massaged conveniently in negative pressure absorption, and various structures can be distributed on the base more uniformly. The heights of the massage post, the bipolar contact electrodes, the annular separator plate and the microcurrent electrodes gradually increase, which ensures that various functions of the device are not affected to each other, and can be used independently.

According to the multifunctional beauty device and the handpiece of the beauty instrument provided by the present disclosure, the annular separator plate is arranged on the skin contacting base. The negative pressure cavity can be formed between the base and the annular separator plate. The negative pressure air hole connected to the negative pressure generator is provided to form the negative pressure in the negative pressure cavity. Therefore, negative pressure cupping is conducted conveniently. A plurality of bipolar contact electrodes and a plurality of microcurrent electrodes are arranged on a side of the annular separator plate. The bipolar contact electrodes are connected to the bipolar RF generator. The microcurrent electrodes are connected to the low-intermediate frequency amplitude-modulation synthesizer. The bipolar contact electrodes can conduct deep heating on the user for weight reduction. The microcurrent electrodes can conduct microcurrent stimulation on the user for a beauty effect. Meanwhile, with the massage post, when the negative pressure absorptive cupping is conducted, acupoints of the user are squeezed and massaged. With the heating module, before the device is used, the bipolar contact electrodes, the microcurrent electrodes and the massage post can be preheated by the heating module. This improves the body comfort, and prevents the cold metal post from directly contacting the skin of the user to cause the discomfort in a cold period such as winter. Additionally, with the upper outer housing and the handle, the device can be used more conveniently. According to the above solutions, various beauty functions can be synthesized, and the device achieves more functions, a stronger universality and a better use effect.

### BRIEF DESCRIPTION OF THE DRAWINGS

Preferred implementations will be described below in a clear and easy-to-understand manner in conjunction with the drawings to further illustrate the above-mentioned characteristics, technical features, advantages, and implementation methods of the present disclosure.

FIG. 1 is an overall structural schematic view according to an embodiment of the present disclosure;

FIG. 2 is a structural schematic side view of a beauty device according to an embodiment of the present disclosure; and

FIG. 3 is a structural schematic perspective view of a beauty device according to an embodiment of the present disclosure.

In the figures: 1—base, 2—negative pressure air hole, 3—microcurrent electrode, 4—annular separator plate, 5—negative pressure cavity, 6—heating module, 7—first contact electrode, 8—second contact electrode, 9—temperature control switch, 10—upper outer housing, 11—handle seat, 12—grip, 13—arc-shaped gripping member, 14—through hole, and 15—massage post.

### DETAILED DESCRIPTION OF THE EMBODIMENTS

In order to explain the embodiments of the present disclosure or the technical solutions in the prior art more clearly, the specific implementations of the present disclosure will be described below with reference to the drawings. Apparently, the drawings in the following description show merely some embodiments of the present disclosure, and other drawings and other implementations may be derived from these drawings by those of ordinary skill in the art without creative efforts.

In order to keep the drawings concise, only components related to the present disclosure are schematically illustrated in each drawing, which do not represent its actual structure as a product. Further, for the purpose of a better understanding, only one of components having the same structure or function is schematically shown or marked in some drawings. In the description of the present disclosure, “one” not only means “only one”, but also means “more than one”.

### Embodiment 1

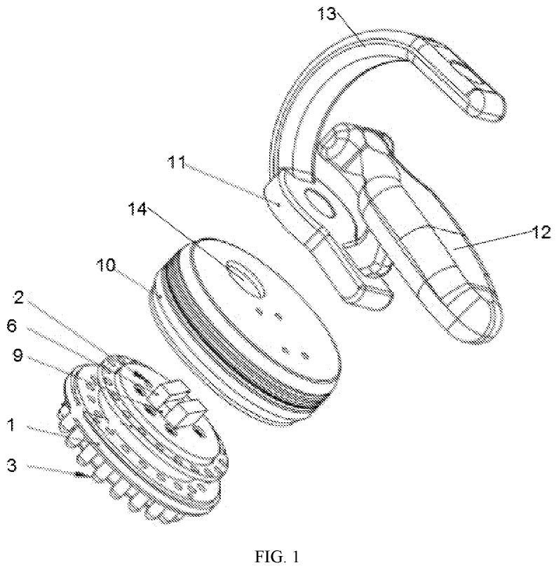

According to an embodiment of the present disclosure, as shown in FIG. 1 to FIG. 3, the present disclosure provides a multifunctional beauty device, including base 1, negative pressure air hole 2, a plurality of bipolar contact electrodes, a plurality of microcurrent electrodes 3, heating module 6, and massage post 15. The base 1 is configured to contact a skin of a human body. There are no limits made on a shape of the base 1. In the embodiments, the base 1 is preferably of a circular shape. In other embodiments, the base 1 may further be of a square shape, an elliptical shape, a polygonal shape, etc.

One surface of the base 1 contacting the skin of the human body is provided with annular separator plate 4. Negative pressure cavity 5 is formed between the annular separator plate 4 and the base 1. There are also no limits made on a shape of the annular separator plate 4. The annular separator plate may be of a circular ring shape, a square ring shape, etc. In the embodiment, the annular separator plate 4 is preferably of the circular ring shape concentric with the base 1.

The negative pressure air hole 2 penetrates through the base and communicates with the negative pressure cavity 5. The negative pressure air hole 2 is configured to connect a negative pressure generator (which is the common negative pressure generator in the prior art, and is not shown in the figure). When the device is attached to the skin, and the negative pressure generator is turned on, a negative pressure can be formed in the negative pressure cavity 5. Therefore, the device can be used for negative pressure cupping.

Additionally, a negative pressure sensor may further be arranged in the embodiment. The negative pressure sensor is configured to detect an intensity of the negative pressure in the negative pressure cavity 5, and feed the intensity back to the negative pressure generator, such that the negative pressure generator can control and adjust the negative pressure according to requirements on the negative pressure.

The plurality of bipolar contact electrodes are arranged on one side of the annular separator plate 4 and connected to the base 1. The bipolar contact electrodes are configured to connect a bipolar RF generator (which is the common bipolar RF generator in the prior art, and is not shown in the figure). Therefore, the device can conduct deep heating for weight reduction.

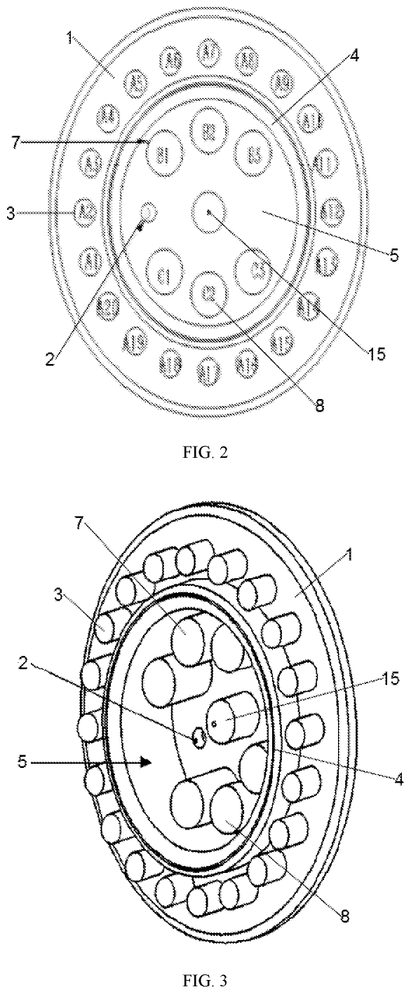

The plurality of microcurrent electrodes 3 (A1-A20 in FIG. 2) are arranged on one side of the annular separator plate 4 and connected to the base 1. The microcurrent electrodes 3 are configured to connect a low-intermediate frequency amplitude-modulation synthesizer (not shown in the figure). Therefore, the device can conduct microcurrent stimulation for a beauty effect.

Additionally, the low-intermediate frequency amplitude-modulation synthesizer in the embodiment is the common low-frequency generator with intermediate-frequency amplitude modulation in the prior art. This makes electrotherapy gentler, improves the body comfort of the user, and does not cause any discomfort to the user.

The massage post 15 is arranged on one side of the annular separator plate 4 and connected to the base 1. The massage post 15 is configured to squeeze and massage acupoints when the negative pressure is sucked and released.

The heating module 6 is arranged on the other surface of the base 1 opposite to the annular separator plate 4 and configured to preheat the bipolar contact electrodes, the microcurrent electrodes 3 and the massage post 15.

With the heating module 6, before the device is used, the bipolar contact electrodes, the microcurrent electrodes 3 and the massage post 15 can be preheated by the heating module 6. This improves the body comfort, and prevents the cold metal post from directly contacting the skin of the user to cause the discomfort in a cold period such as winter.

The annular separator plate 4 is arranged on the skin contacting base 1. The negative pressure cavity 5 can be formed between the base 1 and the annular separator plate 4. The negative pressure air hole 2 connected to the negative pressure generator is provided to form the negative pressure in the negative pressure cavity 5. Therefore, negative pressure cupping is conducted conveniently. A plurality of bipolar contact electrodes and a plurality of microcurrent electrodes 3 are arranged on a side of the annular separator plate 4. The bipolar contact electrodes are connected to the bipolar RF generator. The microcurrent electrodes 3 are connected to the low-intermediate frequency amplitude-modulation synthesizer. The bipolar contact electrodes can conduct deep heating on the user for weight reduction. The microcurrent electrodes 3 can conduct microcurrent stimulation on the user for beauty and stasis removal effects. Meanwhile, with the massage post, when the negative pressure absorptive cupping is conducted, acupoints of the user are squeezed and massaged. With the heating module 6, before the device is used, the bipolar contact electrodes, the microcurrent electrodes 3 and the massage post 15 can be preheated by the heating module. This improves the body comfort, and prevents the cold metal post from directly contacting the skin of the user to cause the discomfort in a cold period such as winter. According to the above solutions, various beauty functions can be synthesized, and the device achieves more functions, a stronger universality and a better use effect.

### Embodiment 2

According to an embodiment of the present disclosure, as shown in FIG. 2 and FIG. 3, on the basis of Embodiment 1, the bipolar contact electrodes are arranged on the inner side of the annular separator plate 4 and distributed annularly.

The bipolar contact electrodes include a plurality of first contact electrodes 7 (B1-B3 in FIG. 2) and a plurality of second contact electrodes 8 (C1-C3 in FIG. 2). The number of the plurality of first contact electrodes is identical to the number of the plurality of second contact electrodes. The plurality of first contact electrodes 7 and the plurality of second contact electrodes 8 are symmetric with respect to the center of the negative pressure cavity 5.

By arranging the bipolar contact electrodes annularly, the bipolar contact electrodes can be used conveniently. Since there is no need to provide too many bipolar contact electrodes, the bipolar contact electrodes can be arranged on the inner side of the annular separator plate 4. The plurality of first contact electrodes 7 and the plurality of second contact electrodes 8 can be symmetric with respect to the center of the negative pressure cavity, which ensures that the bipolar contact electrodes are heated uniformly.

There are three first contact electrodes 7 and three second contact electrodes 8 in the embodiment. In other embodiments, the number of the first contact electrodes 7 and the second contact electrodes 8 can be adjusted as required in use.

Preferably, the microcurrent electrodes 3 are arranged on the outer side of the annular separator plate 4 and distributed annularly and uniformly.

Since there are usually many microcurrent electrodes 3, the microcurrent electrodes 3 can be arranged on the outer side of the annular separator plate 4. Meanwhile, the microcurrent electrodes 3 are distributed annularly and uniformly, which ensures uniform stimulation of a microcurrent on the skin, and improves beauty and stasis removal effects of the microcurrent stimulation.

Further preferably, the massage post 15 is arranged in the center of the negative pressure cavity 5. Heights of the massage post 15, the bipolar contact electrodes, the annular separator plate 4 and the microcurrent electrodes 3 gradually increase.

By providing the massage post 15 in the center of the negative pressure cavity 5, the acupoints of the user can be squeezed and massaged conveniently in negative pressure absorption, and various structures can be distributed on the base 1 more uniformly. The heights of the massage post 15, the bipolar contact electrodes, the annular separator plate 4 and the microcurrent electrodes 3 gradually increase, which ensures that various functions of the device are not affected to each other, and can be used independently.

### Embodiment 3

According to an embodiment of the present disclosure, as shown in FIG. 1, on the basis of any of the foregoing embodiments, the heating module 6 is arranged on the other surface of the base 1 opposite to the annular separator plate 4, and the base 1 is made of a metal material, such that heat generated by the heating module 6 is transferred to the bipolar contact electrodes and the massage post 15.

Since the massage post 15 is made of metal, when the base 1 is also made of the metal material, the heat generated by the heating module 6 can be transferred to the massage post 15 through the base 1. Therefore, the device can be preheated by the heating module 6 before use, which prevents the cold massage post 15 from directly contacting the skin of the user to cause the discomfort in a cold period such as winter.

Additionally, since the bipolar contact electrodes and the microcurrent electrodes 3 are also made of metal, and the heat is transferred through the base 1, the heat generated by the heating module 6 can also be transferred to the bipolar contact electrodes and the microcurrent electrodes 3 through the base 1. Therefore, the bipolar contact electrodes and the microcurrent electrodes 3 can also be preheated by the heating module 6 before use, which improves the body comfort of the user, and prevents the bipolar contact electrodes and the microcurrent electrodes 3 from directly contacting the skin of the user to cause the discomfort in a cold period such as winter.

Preferably, temperature control switch 9 is further arranged between the base 1 and the heating module 6. The temperature control switch 9 is fixed on the base 1 through a thermally conductive silicone pad and connected to the heating module 6. The temperature control switch 9 is disconnected when a temperature of the base 1 reaches a preset temperature, such that the heating module 6 stops heating the base 1.

With the temperature control switch 9, the heating temperature of the base 1 can be monitored in real time, and the human body is not scalded for an overhigh temperature of the metal post. Additionally, the temperature of the temperature control switch is set at 50° C. in the embodiment. Or else, the user is scalded probably by the overhigh temperature. In other embodiments, the monitored temperature can be adjusted as required in use.

### Embodiment 4

According to an embodiment of the present disclosure, on the basis of any of the foregoing embodiments, the bipolar RF generator is connected to a bipolar detection control terminal. The bipolar detection control terminal is configured to initially control the bipolar RF generator to output second energy to the bipolar contact electrodes, and detect a contact condition between the bipolar contact electrodes and a skin. The bipolar detection control terminal controls the bipolar RF generator to output first energy to the bipolar contact electrodes when the bipolar contact electrodes fully contact the skin. The first energy is greater than the second energy.

The bipolar RF generator is connected to the bipolar detection control terminal, such that the bipolar detection control terminal can monitor the contact condition between the bipolar contact electrodes and the skin in real time. Initially, the bipolar detection control terminal controls the bipolar RF generator to output the second energy to the bipolar contact electrodes. When detecting that the bipolar contact electrodes fully contact the skin, the bipolar detection control terminal receives a signal, and controls the bipolar RF generator to output the first energy to the bipolar contact electrodes. When the bipolar contact electrodes contact the skin poorly, the bipolar detection control terminal does not receive the signal, and controls the bipolar RF generator to continuously output the second energy to the bipolar contact electrodes. The first energy refers to normal output energy, while the second energy is weak energy. The first energy is far greater than the second energy. When the bipolar contact electrodes are used to conduct the deep heating on the user for weight reduction, the contact condition between the bipolar contact electrodes and the skin can be found clearly. This prevents the poor contact between the bipolar contact electrodes and the skin, and can further prevent blind increase of energy output to scald the skin when the bipolar contact electrodes contact the skin poorly.

### Embodiment 5

According to an embodiment of the present disclosure, as shown in FIG. 1 to FIG. 3, the present disclosure further provides a handpiece of a beauty instrument, including base 1, negative pressure air hole 2, a plurality of bipolar contact electrodes, a plurality of microcurrent electrodes 3, massage post 15, heating module 6, upper outer housing 10, and a handle. The base 1 is configured to contact a skin of a human body. There are no limits made on a shape of the base 1. In the embodiments, the base 1 is preferably of a circular shape. In other embodiments, the base 1 may further be of a square shape, an elliptical shape, a polygonal shape, etc.

One surface of the base 1 contacting the skin of the human body is provided with annular separator plate 4. Negative pressure cavity 5 is formed between the annular separator plate 4 and the base 1. There are also no limits made on a shape of the annular separator plate 4. The annular separator plate may be of a circular ring shape, a square ring shape, etc. In the embodiment, the annular separator plate 4 is preferably of the circular ring shape concentric with the base 1.

The negative pressure air hole 2 penetrates through the base and communicates with the negative pressure cavity 5. The negative pressure air hole 2 is configured to connect a negative pressure generator (which is the common negative pressure generator in the prior art, and is not shown in the figure). When the device is attached to the skin, and the negative pressure generator is turned on, a negative pressure can be formed in the negative pressure cavity 5. Therefore, the device can be used for negative pressure cupping.

Additionally, a negative pressure sensor may further be arranged in the embodiment. The negative pressure sensor is configured to detect an intensity of the negative pressure in the negative pressure cavity 5, and feed the intensity back to the negative pressure generator, such that the negative pressure generator can control and adjust the negative pressure according to requirements on the negative pressure.

The plurality of bipolar contact electrodes are arranged on one side of the annular separator plate 4 and connected to the base 1. The bipolar contact electrodes are configured to connect a bipolar RF generator (which is the common bipolar RF generator in the prior art, and is not shown in the figure). Therefore, the device can conduct deep heating for weight reduction.

The plurality of microcurrent electrodes 3 (A1-A20 in FIG. 2) are arranged on one side of the annular separator plate 4 and connected to the base 1. The microcurrent electrodes 3 are configured to connect a low-intermediate frequency amplitude-modulation synthesizer (not shown in the figure). Therefore, the device can conduct microcurrent stimulation for a beauty effect.

Additionally, the low-intermediate frequency amplitude-modulation synthesizer in the embodiment is the common low-frequency generator with intermediate-frequency amplitude modulation in the prior art. This makes electrotherapy gentler, improves the body comfort of the user, and does not cause any discomfort to the user.

The massage post 15 is arranged on one side of the annular separator plate 4 and connected to the base 1. The massage post 15 is configured to conduct a massage when the negative pressure is sucked and released. The heating module 6 is arranged on the other surface of the base 1 opposite to the annular separator plate 4 and configured to preheat the bipolar contact electrodes, the microcurrent electrodes 3 and the massage post 15. The device can be preheated by the heating module 6 before use. This improves the body comfort, and prevents the cold metal post from directly contacting the skin of the user to cause the discomfort in a cold period such as winter.

The upper outer housing 10 is connected to the other surface of the base opposite to the annular separator plate 4 and sleeved outside the heating module 6. The handle is connected to the upper outer housing 10.

The annular separator plate 4 is arranged on the skin contacting base 1. The negative pressure cavity 5 can be formed between the base 1 and the annular separator plate 4. The negative pressure air hole 2 connected to the negative pressure generator is provided to form the negative pressure in the negative pressure cavity 5. Therefore, negative pressure cupping is conducted conveniently. A plurality of bipolar contact electrodes and a plurality of microcurrent electrodes 3 are arranged on a side of the annular separator plate 4. The bipolar contact electrodes are connected to the bipolar RF generator. The microcurrent electrodes 3 are connected to the low-intermediate frequency amplitude-modulation synthesizer. The bipolar contact electrodes can conduct deep heating on the user for weight reduction. The microcurrent electrodes 3 can conduct microcurrent stimulation on the user for beauty and stasis removal effects. Meanwhile, with the massage post 15, when the negative pressure absorptive cupping is conducted, acupoints of the user are squeezed and massaged. With the heating module 6, before the device is used, the bipolar contact electrodes, the microcurrent electrodes 3 and the massage post 15 can be preheated by the heating module 6. This improves the body comfort, and prevents the cold metal post from directly contacting the skin of the user to cause the discomfort in a cold period such as winter. Additionally, with the upper outer housing 10 and the handle, the device can be used more conveniently. According to the above solutions, various beauty functions can be synthesized, and the device achieves more functions, a stronger universality and a better use effect.

### Embodiment 6

According to an embodiment of the present disclosure, as shown in FIG. 1, on the basis of Embodiment 5, the handle includes handle seat 11 connected to the upper outer housing 10. Grip 12 is arranged on one side of a top of the handle seat 11. Arc-shaped gripping member 13 is arranged on the other side of the top of the handle seat 11.

Through holes 14 are respectively formed in a surface of the upper outer housing 10 and a surface of the handle seat 11. The through holes 14 are configured to allow a negative pressure air tube and a line to pass through.

By providing the handle seat 11, and providing the grip 12 and the arc-shaped gripping member 13 on the handle seat 11, the device can be held conveniently by a single hand or both hands. By forming the through holes 14 in the upper outer housing 10 and the handle seat 11, the negative pressure air tube connected to the negative pressure air hole 2 and the negative pressure generator, as well as the line connecting the bipolar RF generator and the low-intermediate frequency amplitude-modulation synthesizer, can be provided conveniently, and the negative pressure air tube, the line and the like are not twined to affect use of the handpiece.

### Embodiment 7

According to an embodiment of the present disclosure, as shown in FIG. 2 and FIG. 3, on the basis of Embodiment 5 or Embodiment 6, the bipolar contact electrodes are arranged on the inner side of the annular separator plate 4 and distributed annularly. The microcurrent electrodes 3 are arranged on the outer side of the annular separator plate 4 and distributed annularly and uniformly. The massage post 15 is arranged in the center of the negative pressure cavity 5. Heights of the massage post 15, the bipolar contact electrodes, the annular separator plate 4 and the microcurrent electrodes 3 gradually increase.

By arranging the bipolar contact electrodes annularly, the bipolar contact electrodes can be used conveniently. Since there is no need to provide too many bipolar contact electrodes, the bipolar contact electrodes can be arranged on the inner side of the annular separator plate 4. Since there are usually many microcurrent electrodes 3, the microcurrent electrodes 3 can be arranged on the outer side of the annular separator plate 4. Meanwhile, the microcurrent electrodes 3 are distributed annularly and uniformly, which ensures uniform stimulation of a microcurrent on the skin, and improves beauty and stasis removal effects of microcurrent stimulation. By providing the massage post 15 in the center of the negative pressure cavity 5, the acupoints of the user can be squeezed and massaged conveniently in negative pressure absorption, and various structures can be distributed on the base 1 more uniformly. The heights of the massage post 15, the bipolar contact electrodes, the annular separator plate 4 and the microcurrent electrodes 3 gradually increase, which ensures that various functions of the device are not affected to each other, and can be used independently.

It should be noted that the above embodiments can be freely combined as required. The above are merely preferred implementations of the present disclosure. It should be noted that several improvements and modifications may further be made by a person of ordinary skill in the art without departing from the principle of the present disclosure, and such improvements and modifications should also be deemed as falling within the protection scope of the present disclosure.

What is claimed is:

1. A multifunctional beauty device, comprising:
a base, wherein a first surface of the base is provided with an annular separator plate, and a negative pressure cavity is formed between the annular separator plate and the base;
a negative pressure air hole penetrating through the base and communicating with the negative pressure cavity, wherein the negative pressure air hole is configured to connect a negative pressure generator to form a negative pressure in the negative pressure cavity;
a plurality of bipolar contact electrodes arranged on an inner side of the annular separator plate and connected to the base, wherein the plurality of bipolar contact electrodes are configured to connect a bipolar radio-frequency (RF) generator;
a plurality of microcurrent electrodes arranged on an outer side of the annular separator plate and connected to the base, wherein the plurality of microcurrent electrodes are configured to connect a low-intermediate frequency amplitude-modulation synthesizer;
a massage post arranged on the outer side of the annular separator plate, connected to the base, and configured to conduct a massage when the negative pressure is sucked and released; and
a heating module arranged on a second surface of the base and configured to preheat the plurality of bipolar contact electrodes, the plurality of microcurrent electrodes and the massage post, wherein the second surface of the base is opposite to the annular separator plate.

2. The multifunctional beauty device according to claim 1, wherein the plurality of bipolar contact electrodes are arranged on the inner side of the annular separator plate and distributed annularly; and
the plurality of bipolar contact electrodes comprise a plurality of first contact electrodes and a plurality of second contact electrodes, wherein a number of the plurality of first contact electrodes is identical to a number of the plurality of second contact electrodes, and the plurality of first contact electrodes and the plurality of second contact electrodes are symmetric with respect to a center of the negative pressure cavity.

3. The multifunctional beauty device according to claim 1, wherein the plurality of microcurrent electrodes are arranged on the outer side of the annular separator plate and distributed annularly and uniformly.

4. The multifunctional beauty device according to claim 1, wherein the massage post is arranged in a center of the negative pressure cavity; and
heights of the massage post, the plurality of bipolar contact electrodes, the annular separator plate and the plurality of microcurrent electrodes gradually increase.

5. The multifunctional beauty device according to claim 1, wherein the heating module is arranged on the second surface of the base, and the base is made of a metal material, such that heat generated by the heating module is transferred to the plurality of bipolar contact electrodes, the plurality of microcurrent electrodes and the massage post.

6. The multifunctional beauty device according to claim 5, wherein a temperature control switch is arranged between the base and the heating module;
the temperature control switch is fixed on the base through a thermally conductive silicone pad and connected to the heating module; and
the temperature control switch is disconnected when a temperature of the base reaches a preset temperature, such that the heating module stops heating the base.

7. The multifunctional beauty device according to claim 1, wherein the bipolar RF generator is connected to a bipolar detection control terminal; and the bipolar detection control terminal is configured to initially control the bipolar RF generator to output second energy to the plurality of bipolar contact electrodes, and detect a contact condition between the plurality of bipolar contact electrodes and a skin; and
the bipolar detection control terminal controls the bipolar RF generator to output first energy to the plurality of bipolar contact electrodes when the plurality of bipolar contact electrodes fully contact the skin, wherein the first energy is greater than the second energy.

8. A handpiece of a beauty instrument, comprising:
a base, wherein a first surface of the base is provided with an annular separator plate, and a negative pressure cavity is formed between the annular separator plate and the base;
a negative pressure air hole penetrating through the base and communicating with the negative pressure cavity, wherein the negative pressure air hole is configured to connect a negative pressure generator to form a negative pressure in the negative pressure cavity;
a plurality of bipolar contact electrodes arranged on an inner side of the annular separator plate and connected to the base, wherein the plurality of bipolar contact electrodes are configured to connect a bipolar RF generator;
a plurality of microcurrent electrodes arranged on an outer side of the annular separator plate and connected to the base, wherein the plurality of microcurrent electrodes are configured to connect a low-intermediate frequency amplitude-modulation synthesizer;
a massage post arranged on the outer side of the annular separator plate, connected to the base, and configured to conduct a massage when the negative pressure is sucked and released;
a heating module arranged on a second surface of the base and configured to preheat the plurality of bipolar contact electrodes, the plurality of microcurrent electrodes and the massage post, wherein the second surface of the base is opposite to the annular separator plate;
an upper outer housing connected to the second surface of the base and sleeved outside the heating module; and
a handle connected to the upper outer housing.

9. The handpiece of the beauty instrument according to claim 8, wherein the handle comprises a handle seat connected to the upper outer housing, wherein a grip is arranged on a first side of a top of the handle seat, and an arc-shaped gripping member is arranged on a second side of the top of the handle seat; and
through holes are respectively formed in a surface of the upper outer housing and a surface of the handle seat, and the through holes are configured to allow a negative pressure air tube and a line to penetrate through.

10. The handpiece of the beauty instrument according to claim 8, wherein the plurality of bipolar contact electrodes are arranged on the inner side of the annular separator plate and distributed annularly;
the plurality of microcurrent electrodes are arranged on the outer side of the annular separator plate and distributed annularly and uniformly;
the massage post is arranged in a center of the negative pressure cavity; and
heights of the massage post, the plurality of bipolar contact electrodes, the annular separator plate and the plurality of microcurrent electrodes gradually increase.
